(19) United States
(12) Patent Application Publication
(10) Pub. No.: US 2021/0292304 A1
(43) Pub. Date: Sep. 23, 2021

(54) DEUTERATED MGL-3196 COMPOUND AND USE THEREOF

(71) Applicant: HINOVA PHARMACEUTICALS INC., Chengdu, Sichuan (CN)

(72) Inventors: Wu DU, Chengdu, Sichuan (CN); Yu LI, Chengdu, Sichuan (CN); Haibo LI, Chengdu, Sichuan (CN); Yuanwei CHEN, Chengdu, Sichuan (CN); Chengzhi ZHANG, Chengdu, Sichuan (CN); Xinghai LI, Chengdu, Sichuan (CN)

(21) Appl. No.: 17/255,231

(22) PCT Filed: Jun. 21, 2019
(86) PCT No.: PCT/CN2019/092386

§ 371 Date: May 12, 2021

(30) Foreign Application Priority Data
Jun. 22, 2018 (CN) 201810651269.4

(51) Int. Cl. C07D 403/12

(52) U.S. Cl. CPC C07D 403/12; C07B 2200/05

(57) ABSTRACT

Disclosed are a compound as shown in formula (I) or optical isomers, pharmaceutically acceptable salts, prodrugs, hydrates or solvates thereof, wherein R¹-R¹⁰ are independently selected from H and D, respectively, and not all are H. Compared to the undeuterated control compound MGL3 196, the compound of formula (I) or the optical isomers, pharmaceutically acceptable salts, prodrugs, hydrates or solvates thereof has/have better agonistic activity on thyroid hormone receptor p (THR-p), has/have a longer half-life and a lower clearance rate, has/have better metabolic stability and pharmacokinetic properties, and has/have excellent application prospects in the preparation of THR-p agonists and drugs for treating indications to which THR-p agonists are applicable, including dyslipidemia, hypercholesterolemia, nonalcoholic steatohepatitis (NASH) and nonalcoholic fatty liver disease (NAFLD).

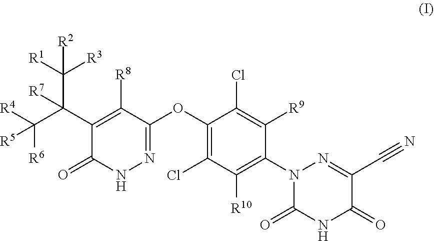

### TECHNICAL FIELD

The present invention relates to a deuterated MGL-3196 compound and the use thereof.

### BACKGROUND ART

MGL-3196 is a highly selective thyroid hormone receptor β (THR-β) agonist with an EC₅₀ value of 0.21 μM, having a structural formula of

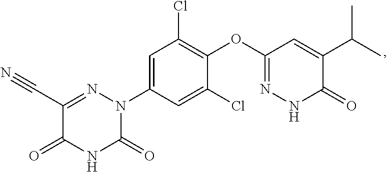 [SMILES: CC(C)c1cc(Oc2c(Cl)cc(-n3nc(C#N)c(=O)[nH]c3=O)cc2Cl)n[nH]c1=O]

and currently, it is undergoing late-stage clinical trials, showing the effects on dyslipidemia, hypercholesterolemia, and non-alcoholic steatohepatitis (NASH).

Deuterated drugs denote the replacement of part of the hydrogen atoms in drug molecules with deuterium. Since the shape and the volume of deuterium are close to those of hydrogen in the drug molecule, deuterated drugs generally retain the biological activity and selectivity of the original drug. Because C-D bond is more stable than C—H bond, C-D bond is less likely to be broken during the chemical reaction of deuterated drugs, and its half-life may be prolonged.

However, due to the complex metabolic processes in biological systems, the pharmacokinetic properties of drugs in organisms are influenced by many factors, and they also exhibit corresponding complexity. Compared with the corresponding non-deuterated drugs, the changes in the pharmacokinetic properties of deuterated drugs show great chance and unpredictability. Deuteration at certain sites not only cannot prolong the half-life, but may shorten it (Scott L. Harbeson, Roger D. Tung. Deuterium in Drug Discovery and Development, P405-406.), and affect pharmacokinetic properties; on the other hand, hydrogens at certain positions of drug molecules are not easily deuterated due to steric hindrance and other reasons. Therefore, the deuteration of drugs is not arbitrary, and the sites that can be deuterated are unpredictable.

In the present invention, a class of deuterated drugs, that have good pharmacokinetic properties, lower dosage, and lower toxic and side effects, are expected to be obtained by deuterating MGL-3196 compound.

### CONTENT OF THE INVENTION

The purpose of the present invention is to provide a class of deuterated MGL-3196 drugs with good pharmacokinetic properties, low toxic and side effects, and good metabolic stability.

The present invention first provides the compound of formula (I) or an optical isomer, a pharmaceutically acceptable salt, a prodrug, a hydrate or a solvate thereof:

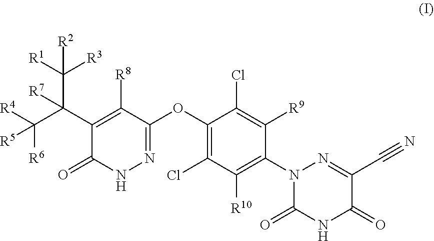

Wherein, R¹-R¹⁰ are each independently selected from H and D, and not all H.

Further, said compound has the structure of formula (II):

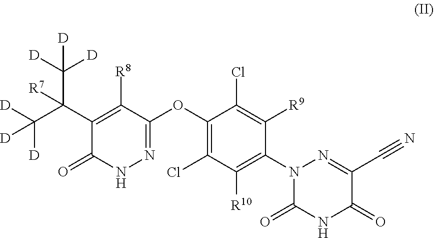

Wherein, R⁷-R¹⁰ are each independently selected from H and D.

Further, said compound has the structure of formula (III):

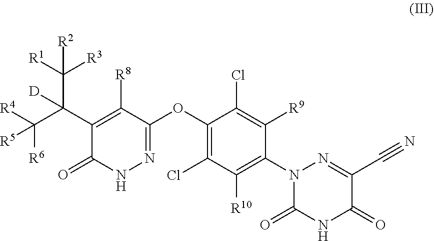

Wherein, R¹-R⁶ and R⁸-R¹⁰ are each independently selected from H and D.

Further, said compound has the structure of formula (IV):

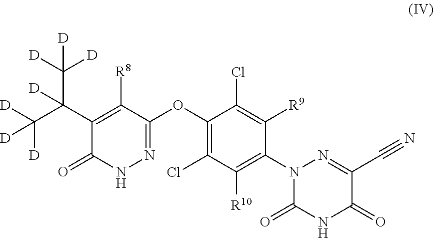

Wherein, R⁸-R¹⁰ are each independently selected from H and D.

Further, said compound has the structure of formula (V):

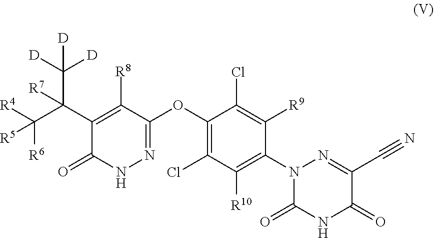

Wherein, R⁴-R¹⁰ are each independently selected from H and D.

Further, said compound has the structure of formula (VI):

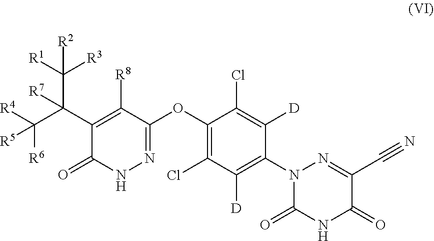

Wherein, R¹-R⁸ are each independently selected from H and D.

Further, said compound is selected but not limited to one of the following structures:

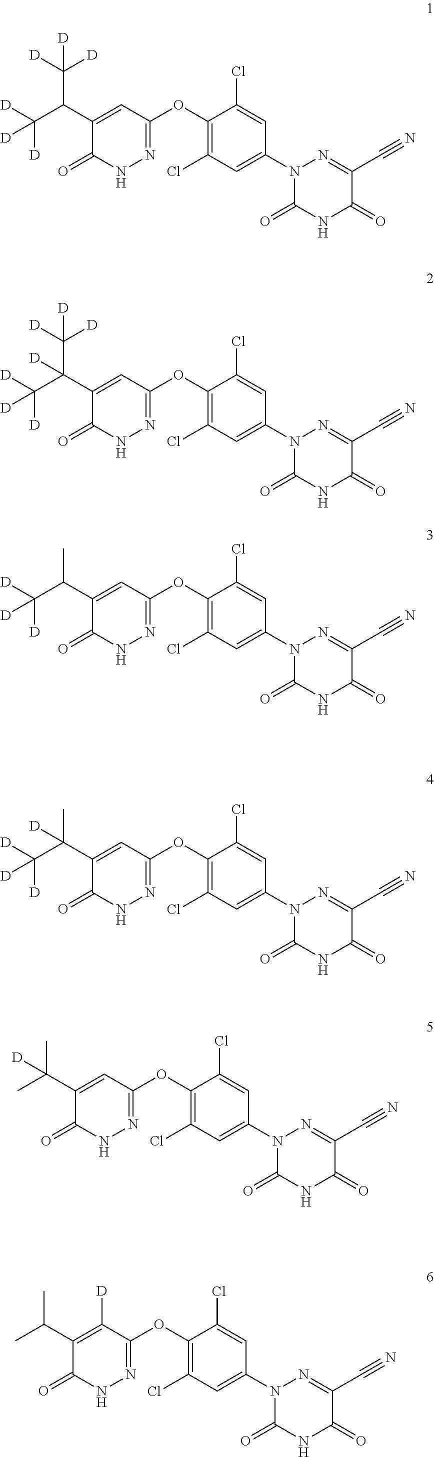 [SMILES: [2H]C([2H])([2H])C(c1cc(Oc2c(Cl)cc(-n3nc(C#N)c(=O)[nH]c3=O)cc2Cl)n[nH]c1=O)C([2H])([2H])[2H] ; [2H]C([2H])([2H])C([2H])(c1cc(Oc2c(Cl)cc(-n3nc(C#N)c(=O)[nH]c3=O)cc2Cl)n[nH]c1=O)C([2H])([2H])[2H] ; [2H]C([2H])([2H])C(C)c1cc(Oc2c(Cl)cc(-n3nc(C#N)c(=O)[nH]c3=O)cc2Cl)n[nH]c1=O ; [2H]C([2H])([2H])C([2H])(C)c1cc(Oc2c(Cl)cc(-n3nc(C#N)c(=O)[nH]c3=O)cc2Cl)n[nH]c1=O ; [2H]C(C)(C)c1cc(Oc2c(Cl)cc(-n3nc(C#N)c(=O)[nH]c3=O)cc2Cl)n[nH]c1=O ; [2H]c1c(Oc2c(Cl)cc(-n3nc(C#N)c(=O)[nH]c3=O)cc2Cl)n[nH]c(=O)c1C(C)C]

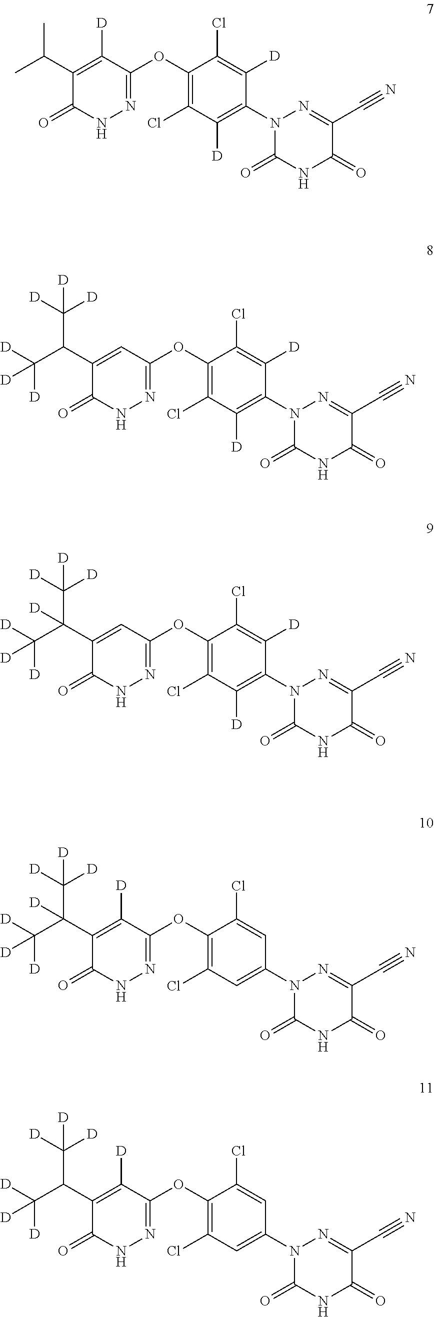

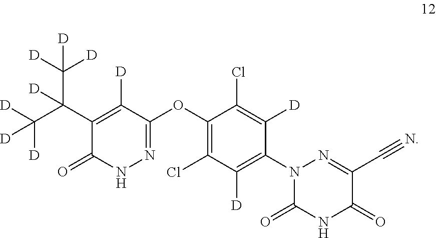 [SMILES: [2H]c1c(Cl)c(Oc2n[nH]c(=O)c(C([2H])(C([2H])([2H])[2H])C([2H])([2H])[2H])c2[2H])c(Cl)c([2H])c1-n1nc(C#N)c(=O)[nH]c1=O]

The present invention further provides the use of the compound mentioned above or an optical isomer, a pharmaceutically acceptable salt, a prodrug, a hydrate or a solvate thereof in the preparation of the drugs for reducing cholesterol as well as treating dyslipidemia and non-alcoholic steatohepatitis (NASH).

Further, said drug is those for treatment of familial hypercholesterolemia, non-alcoholic steatohepatitis (NASH), and non-alcoholic fatty liver disease (NAFLD). The present invention further provides the use of the compound mentioned above or an optical isomer, a pharmaceutically acceptable salt, a prodrug, a hydrate or a solvate thereof in the preparation of THR-β agonist.

The present invention further provides a drug for lowering cholesterol as well as treating dyslipidemia and non-alcoholic fatty liver, that is a preparation obtained by using the compound mentioned above or an optical isomer, a pharmaceutically acceptable salt, a prodrug, a hydrate or a solvate thereof as active ingredients, with the addition of pharmaceutically acceptable excipients.

As used herein, “deuterated” means the replacement of one or more hydrogens in a compound or a group with deuterium. Deuteration can be mono-, di-, poly-, or fully-substituted. In another preferred example, the deuterium isotope content at the deuterium substitution position is more than the natural deuterium isotope content (0.015%), preferably more than 50%, preferably more than 75%, preferably more than 95%, preferably more than 97%, preferably more than 99%, and preferably more than 99.5%.

As used herein, the term “compound of the present invention” means the compound of formula (I). The term also includes various optical isomers, pharmaceutically acceptable salts, prodrugs, hydrates or solvates of compound of formula (I).

The active ingredient mentioned herein denotes any substance or substance mixture used in the manufacture of drug. Such substances have pharmacological activity or other direct effects in the diagnosis, treatment, symptom alleviation, treatment or prevention of diseases, or can influence the function or structure of the body.

The pharmaceutically acceptable excipients described herein have certain physiological activities, but the addition of the ingredients will not change the dominant position of the above-mentioned pharmaceutical composition in the course of disease treatment, but only exert auxiliary functions. These auxiliary functions are only the use of the known activity of the ingredients, that is a commonly used auxiliary treatment in the medical field. If the aforementioned auxiliary components are used in conjunction with the pharmaceutical composition of the present invention, they should still fall within the protection scope of the present invention.

Compared with the non-deuterated control compound MGL3196, the compound provided in the present invention or an optical isomer, a pharmaceutically acceptable salt, a prodrug, a hydrate or a solvate thereof have better agonistic effect on thyroid hormone receptor R (THR-β), longer half-life, lower clearance rate, better metabolic stability and pharmacokinetic properties. The application prospects in the preparation of THR-β agonists and drugs for treatment of dyslipidemia, hypercholesterolemia, and non-alcoholic steatohepatitis (NASH) are excellent.

Obviously, based on above content of the present invention, according to the common technical knowledge and the conventional means in the field, without department from above basic technical spirits, other various modifications, alternations or changes can further be made.

By following specific examples of said embodiments, above content of the present invention is further illustrated. But it should not be construed that the scope of above subject of the present invention is limited to following examples. The techniques realized based on above content of the present invention are all within the scope of the present invention.

### EXAMPLES

The starting materials and equipment used in the present invention are all known products and can be obtained by purchasing commercially available products.

### Example 1 Synthesis of 2-(3,5-dichloro-4-((5-(1,1,1,3,3,3-hexadeuteropropan-2-yl)-6-oxo-1,6-dihydro-pyridazin3-yl)oxy)phenyl)-3,5-dioxo-2,3,4,5-tetrahydro-1,2,4-triazine-6-carbonitrile (1)

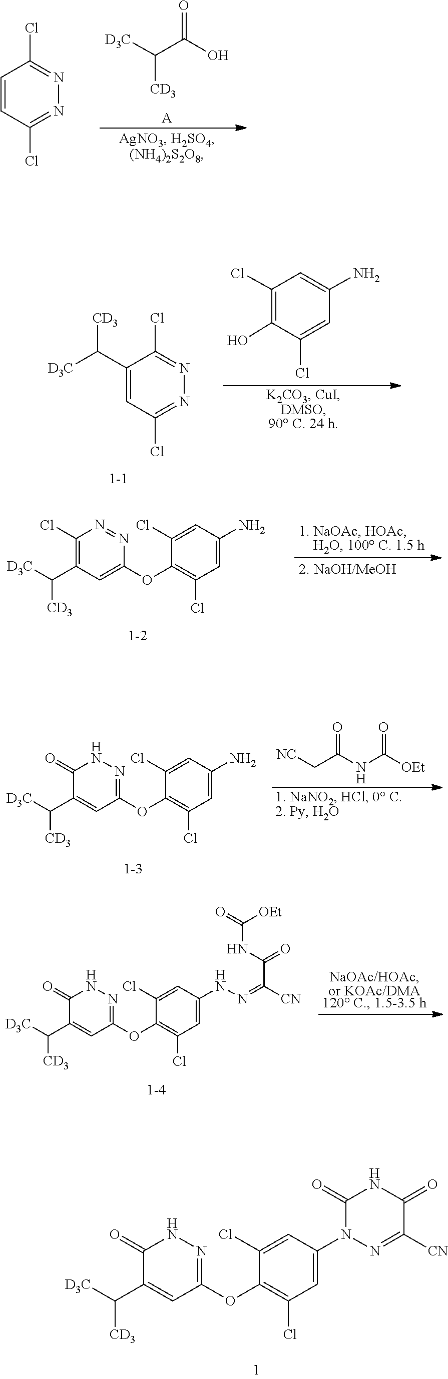

Using literatural methods (Canadian Journal of Chemistry, 2014, 92, 305), 2-trideuteromethyl-3,3,3-trideuteropropionic acid A was prepared.

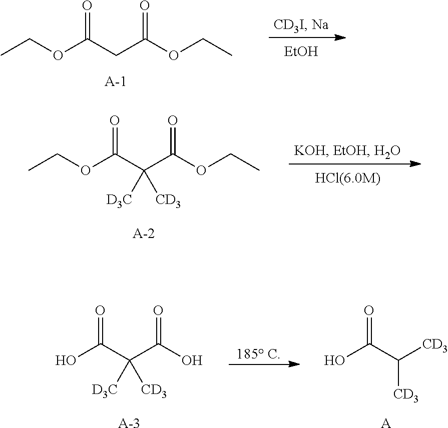

350 mL ethanol was measured and poured into a 500 mL three-necked round bottom flask, then stirred at room temperature. After that, flaky Na (9.9 g, 430.79 mmol) was slowly added to the system in batches, and when the system was completely clear, the system was transferred to an oil bath to continue heating and stirring. When the internal temperature of the system rose to 70° C., diethyl malonate (30 g, 187.30 mmol) was added dropwise to the system. After addition, the mixture was stirred for 15 min under the same temperature, to which was added iodomethane-d₃ (57 g, 393.33 mmol) dropwise, and the dropping rate was controlled to maintain the reflux state in the system. After addition, the system was continually allowed to stir and react in an oil bath at 90° C. After 4 h, TLC was used to monitor the complete consumption of starting materials. The oil bath was removed, and the system was cooled to room temperature, and the solvent was removed by rotary evaporation to obtain a crude product, which was separated by column chromatography to obtain diethyl 2,2-dideuteromethylmalonate A-2 (23 g), as colorless and transparent oily liquid. Yield: 63.2%. MS (ESI) m/z 195.3 [M+H]⁺.

Diethyl-2,2-dideuteromethylmalonate A-2 (12.0 g, 6.18 mmol) was weighed and moved into a 250 mL three-necked round-bottom flask, to which was added 12.5 mL ethanol, and the reaction was stirred at room temperature to dissolve A-2 and allow the solution become clear. Subsequently, 12.5 mL aqueous KOH solution (16.8 g, 30 mmol) was added to the system. After addition, the system was moved to an oil bath at 85° C., and the reaction was continually heated and stirred. After 1 h, heating was stopped, and the system was naturally cooled to room temperature and stirred overnight.

The next day, the system was placed in an ice water bath to cool down under stirring. When the internal temperature of the system was reduced to 0° C., HCl (6.0 M) solution was added dropwise to the system to adjust the pH value of the system to about 2, and the dropping rate was controlled to make the internal temperature not exceed 5° C. After that, the solvent was concentrated under reduced pressure, and then toluene was added to co-evaporate with the water in the system, that was repeated several times until the system appeared completely dry. Then, ACN (100 mL) was added to the system, and the system was placed in an oil bath at 85° C., refluxed under stirring for 15 min, and filtered while hot. The filter cake was collected, and this operation was repeated three times. The filtrate was combined, and the solvent was removed by rotary evaporation to obtain a brown solid, that was placed in a 50 mL single-necked round-bottom flask, to which was added toluene (15 mL). The mixture was stirred at room temperature to make a slurry, and after 20 min, the operation of suction filtration was performed. The filter cake was rinsed with a small amount of toluene (5 mL) several times and dried under vacuum, to obtain 2,2-bis(trideuteromethyl)malonic acid A-3 (4.7 g) as white solid. Yield: 55.1%. MS (ESI) m/z 156.2 [M+H₂O]⁺.

2,2-Dideuteromethylmalonic acid A-3 (3.0 g, 34 mmol) was weighed and moved into a 25 mL single-necked round-bottom flask, and then the system was placed in an oil bath at 185° C. and stirred. When it was in a completely molten state, the system was stirred at the temperature of 185° C. for 30 min. The heating was stopped, and the system was naturally cooled to room temperature. The system was distilled under reduced pressure to obtain a colorless transparent liquid 2-trideuteromethyl-3,3,3-trideuteropropionic acid A (1.5 g). Yield: 73.4%. ¹H NMR (400 MHz, DMSO-d₆) δ 11.82 (br, 1H), 2.38 (s, 1H).

### Step 1: Synthesis of 3,6-dichloro-4-(1,1,1,3,3,3-hexadeuteropropan-2-yl)pyridazine (compound 1-1)

2-Trideuteromethyl-3,3,3-trideuteropropionic acid A (1.4 g, 15 mmol) was weighed and placed into a 100 mL three-necked round-bottom flask, to which was added 20 mL water, and stirred at room temperature to dissolve A and allow the solution become clear. Then, 3,6-dichloropyridazine (2.2 g, mmol) was added to the system, and the reaction was stirred at room temperature. Then, silver nitrate (2.5 g, 15 mmol) was added to the system, after which the system was transferred to an oil bath, heated and stirred for reaction. When the internal temperature of the system rose to 50° C., to which was dropwise added concentrated sulfuric acid (3.5 mL). After addition, the system was stirred at the temperature of 50° C. for 10 min. Then, when the internal temperature of the system rose to 60° C., 6 mL aqueous solution of ammonium persulfate (10.3 g, 45 mmol) was added dropwise to the system. When the internal temperature of the system rose to 70° C., it was allowed to react and stirred at this temperature for 30 minutes. The heating was stopped, and the system was naturally cooled to room temperature. Then, the system was transferred to an ice water bath to cool down under stirring. After 15 min, NaOH (6.0 M) solution was added dropwise to the system to adjust pH to about 8. Ethyl acetate (20 mL) was added to the system, and the mixture was stirred vigorously and stood for layering and separating. The aqueous phase was back extracted with ethyl acetate (10 mL×3), the organic phase was combined, and then successively washed with water (10 mL×3) and saturated brine (20 mL), followed by drying over anhydrous sodium sulfate. The solvent was concentrated under reduced pressure to obtain a crude product, which was separated by column chromatography to obtain 3,6-dichloro-4-(1,1,1,3,3,3-hexadeuteropropan-2-yl) pyridazine (compound 1-1, 1.7 g) as a white solid. Yield: 58%. MS (ESI) m/z 197.2 [M+H]⁺. ¹H NMR (400 MHz, DMSO-d₆) δ 7.98 (d, J=0.8 Hz, 1H), 3.12 (s, 1H).

### Step 2: Synthesis of 3,5-dichloro-4-((6-chloro-5-(1,1,1,3,3,3-hexadeuteropropan-2-yl) pyridazin-3-yl)oxy)aniline (compound 1-2)

3,6-Dichloro-4-(1,1,1,3,3,3-hexadeuteropropan-2-yl)pyridazine (1.7 g, 8.46 mmol) was weighed and moved into a 100 mL three-necked round-bottom flask, to which was added 10 mL dimethyl sulfoxide, and the mixture was stirred at room temperature to dissolve and become clear. The operation of purging argon was performed and repeated ten times to ensure an inert gas atmosphere in the system. Subsequently, 4-amino-2,6-dichlorophenol (1.5 g, 8.46 mmol), anhydrous potassium carbonate (4.7 g, 33.84 mmol), and cuprous iodide (967.5 mg, 5.08 mmol) were sequentially added to the system. After addition, the system was placed in an oil bath at 90° C., and the reaction was heated and stirred overnight. After 24 h, the disappearance of starting materials was monitored. Then, heating was stopped, and the system was allowed to naturally cool to room temperature. To the system, were added ethyl acetate (20 mL) and water (20 mL), and the reaction was stirred vigorously, and then stood to separate the layers. The aqueous phase was back-extracted with ethyl acetate (20 mL×3), and the organic phase was combined and successively washed with water (10 mL×3) and saturated brine (20 mL), then dried on anhydrous sodium sulfate. The solvent was concentrated under reduced pressure to obtain a crude product, which was then separated by column chromatography to obtain a solid (1.7 g). Yield: 58.2%. MS (ESI) m/z 338.7 [M+H]⁺.

### Step 3: Synthesis of 6-(4-amino-2,6-dichlorophenoxy)-4-(1,1,1,3,3,3-hexadeuteropropan-2-yl) pyridazin-3(2H)-one (compound 1-3)

Compound 1-2 (1.0 g, 2.95 mmol) was weighed and placed in a 100 mL three-necked round-bottom flask, to which was added glacial acetic acid (30 mL), and the mixture was stirred at room temperature. Then, anhydrous sodium acetate (847 mg, 10.33 mmol) was added to the system. After addition, the system was placed in an oil bath at 105° C., stirred, and refluxed for reaction. After 24 h, heating was stopped, and the system was allowed to naturally cool to room temperature. The solvent was removed by rotary evaporation, water (150 mL) was added to the system, and the system was then transferred to an ice-water bath to cool down under stirring. When the internal temperature of the system dropped to 5° C., sodium hydroxide (1.0 M) solution was added dropwise to adjust the pH value to about 9. Then, to the system was added ethyl acetate (100 mL), and the mixture was stirred vigorously, and then stood to separate the layers. The aqueous phase was back-extracted with ethyl acetate (50 mL×2), and the organic phase was combined and washed once with water (30 mL) and brine (30 mL), respectively, followed by drying over anhydrous sodium sulfate and concentrating under reduced pressure, to obtain a pale yellow solid. Methanol (20 mL) and NaOH (1.0 M) solution (20 mL) were sequentially added to a 100 mL three-necked round-bottom flask containing the solid. After addition, the system was transferred to an oil bath at 105° C. and refluxed for reaction. After 17 h, heating was stopped, the oil bath was removed, and the system was allowed to return to room temperature. The solvent was removed by rotary evaporation, and then ethyl acetate (160 mL) and water (100 mL) were added. The mixture was stirred vigorously, and then stood to separate the layers. The aqueous layer was back-extracted with ethyl acetate (25 mL×2), and the organic layers were combined, then washed successively with water (20 mL×2) and saturated brine (20 mL), followed by drying with anhydrous sodium sulfate and removing the solvent by rotary evaporation, to obtain a crude product, which was then separated by column chromatography to obtain compound 1-3 (823 mg) as solid. Yield: 87%. MS (ESI) m/z 320.2 [M+H]⁺. ¹H NMR (400 MHz, DMSO-d₆) δ 12.13 (s, 1H), 7.27 (d, J=0.8 Hz, 1H), 6.66 (s, 2H), 5.62 (s, 2H), 2.98 (s, 1H).

### Step 4: Synthesis of ethyl (2-cyano-2-(2-(3,5-dichloro-4-((5-(1,1,1,3,3,3-hexadeuteropropan-2-yl)-6-oxo-1,6-dihydro-pyridazin-3-yl)oxy)phenyl)hydrono)acetyl)carbamate (compounds 1-4)

Compound 1-3 (134 mg, 0.42 mmol) was weighed and moved to a 25 mL single-necked round-bottom flask, to which was added water (5.6 mL), and the mixture was stirred at room temperature. Then, concentrated hydrochloric acid (2.8 mL) was added to the system. After addition, the system was placed in an ice water bath to cool down under stirring. When the internal temperature of the system dropped to 0° C., 0.4 mL aqueous solution of sodium nitrite (36.5 mg, 0.53 mmol) was added dropwise to the system. Then, the system was continually stirred and reacted 30 min at the temperature of 0° C. In addition, N-cyanoacetylurethane (71 mg, 0.46 mmol) was weighed and placed in a 25 mL single-necked round-bottom flask, to which were added water (9.4 mL) and pyridine (2.8 mL), and the mixture was stirred at room temperature to dissolve and make it clear. Then, the system was moved to an ice-water bath to continue cooling and stirring for 30 min. The diazotization reaction solution was slowly added dropwise to the system containing N-cyanoacetylurethane, and the dropping rate was controlled so that the internal temperature of the system did not exceed 5° C. After addition, the system was still left in the ice-water bath to react under stirring. After 1 h, TLC was used to monitor the completion of the reaction. The system was subjected to the operation of suction filtration, the filter cake was rinsed several times with a small amount of water, and then rinsed several times with n-hexane. After drying, an orange solid (124 mg) was obtained. Without further purification, it was directly used in the next reaction. Yield: 60.8%. MS (ESI) m/z 487.1 (M+H)⁺. ¹H NMR (400 MHz, DMSO-d₆) δ 12.22 (s, 1H), 12.14 (br, 1H), 10.90 (s, 1H), 7.99 (s, 2H), 7.37 (d, J=0.8 Hz, 1H), 4.23-4.17 (q, J=14.0, 7.2 Hz, 2H), 3.00 (s, 1H), 1.29-1.25 (t, J=7.2 Hz, 3H).

### Step 5: Synthesis of 2-(3,5-dichloro-4-((5-(1,1,1,3,3,3-hexadeuteropropan-2-yl)-6-oxo-1,6-dihydro-pyridazin3-yl)oxy)phenyl)-3,5-dioxo-2,3,4,5-tetrahydro-1,2,4-triazine-6-carbonitrile (compound 1)

Compound 1-5 (124 mg, 0.25 mmol) was weighed and placed in a 25 mL single-necked round-bottom flask, to which was added glacial acetic acid (3 mL), and the mixture was stirred at room temperature. Then, anhydrous sodium acetate (102.5 mg, 1.25 mmol) was added to the system. After that, the system was moved to an oil bath at 120° C. to increase the temperature under stirring for reaction. After 1 h, TLC was used to monitor the consumption of starting materials. Heating was stopped, the system was cooled to room temperature, and then placed in an ice-water bath to continue cooling and stirring. When the internal temperature of the system dropped to 5° C., ice water was added to the system and then stirred vigorously for 20 min. Subsequently, it was subjected to the operation of suction filtration, the filter cake was rinsed with a small amount of water for several times, and then dissolved in ethyl acetate, followed by drying with anhydrous sodium sulfate and removing the solvent by rotary evaporation to obtain a crude product, which was separated and purified by Pre-TLC to obtain a light orange solid (83 mg). Yield: 74.1%. MS (ESI) m/z 441.0 [M+H]⁺. ¹H NMR (400 MHz, DMSO-d₆) δ 12.2 (br, 1H), 7.78 (s, 2H), 7.43 (d, J=0.8 Hz, 1H), 3.01 (s, 1H).

### Example 2 Synthesis of 2-(3,5-dichloro-4-((5-(heptadeuteroisopropyl-6-oxo-1,6-dihydro-pyridazin3-yl)oxy)phenyl)-3,5-dioxo-2,3,4,5-tetrahydro-1,2,4-triazine-6-carbonitrile (2)

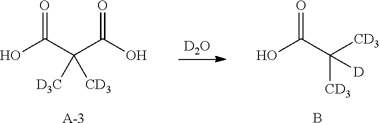

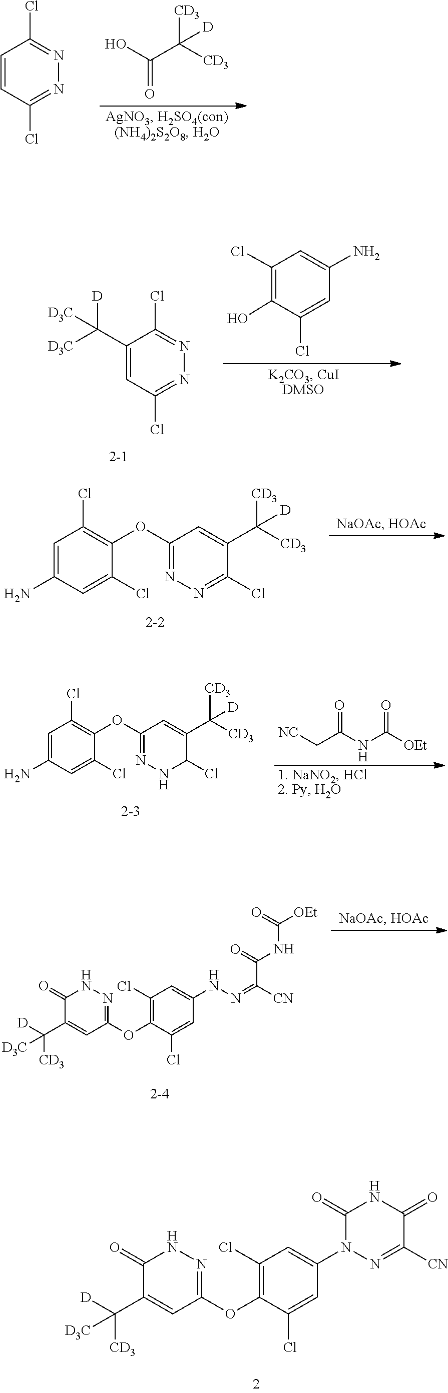

### Synthesis of 2,3,3,3-tetradeuterated-2-(trideuteromethyl)propionic acid (compound B)

2,2-Bis(trideuteromethyl)malonic acid (3 g, 34 mmol) was weighed and placed in a 100 mL single-necked round-bottom flask, to which was added heavy water (15 mL), and the system was moved in a water bath at 60° C., then the solvent was removed by rotatory evaporation. The operation was repeated twice. The substrate mentioned above was transferred to a 35 mL sealed tube, to which was added heavy water (9 mL), and the tube was placed in an oil bath at 160° C. after sealing, and reacted under stirring overnight. After 12 h, the heating was stopped, the system was cooled to room temperature, and the solvent was rotatory evaporated at low temperature to obtain compound B (2.1 g) as a colorless and transparent oily liquid. Without further purification, it was directly used in the next reaction.

Using compound B and 3,6-dichloropyridazine as starting materials, compound 2 was prepared using a method similar to Example 1.

Step 1: Synthesis of 3,6-dichloro-4-(heptadeuteroisopropyl)pyridazine (compound 2-1): Yield: 61.0%. MS (ESI) m/z 198.1 [M+H]⁺.

Step 2: Synthesis of 3,5-dichloro-4-((6-chloro-5-heptadeuteroisopropyl pyridazin-3-yl)oxy) aniline (compound 2-2): Yield: 52.5%. MS (ESI) m/z 339.0 [M+H]⁺.

Step 3: Synthesis of 6-(4-amino-2,6-dichlorophenoxy)-4-heptadeuterioisopropyl pyridazin-3(2H)-one (compound 2-3): Yield: 77.3%. MS (ESI) m/z 321.1 [M+H]⁺.

Step 4: Synthesis of ethyl (2-cyano-2-(2-(3,5-dichloro-4-((5-deuteroisopropyl-6-oxo-1,6-dihydropyridazine)-3-yl)oxy)phenyl)hydrono)acetyl)carbamate (compound 2-4): It was directly used in the next step without further purification. Yield: 91.3%. MS (ESI) m/z 488.0 [M+H]⁺.

Step 5: Synthesis of 2-(3,5-dichloro-4-((5-heptadeuteroisopropyl-6-oxo-1,6-dihydropyridazine-3-yl)oxy)phenyl)-3,5-dioxo-2,3,4,5-tetrahydro-1,2,4-triazine-6-carbonitrile (compound 2-5): Yield 95.3%. MS (ESI) m/z 442.0 [M+H]⁺. ¹H NMR (400 MHz, DMSO-d₆) δ 13.30 (br, 1H), 12.25 (s, 1H), 7.78 (s, 2H), 7.45 (s, 1H).

### Example 3 Synthesis of 2-(3,5-dichloro-4-((5-(1,1,1-heptadeuteriopropan-2-yl)-6-oxo-1,6-dihydro-pyridazin3-yl)oxy)phenyl)-3,5-dioxo-2,3,4,5-tetrahydro-1,2,4-triazine-6-carbonitrile (3)

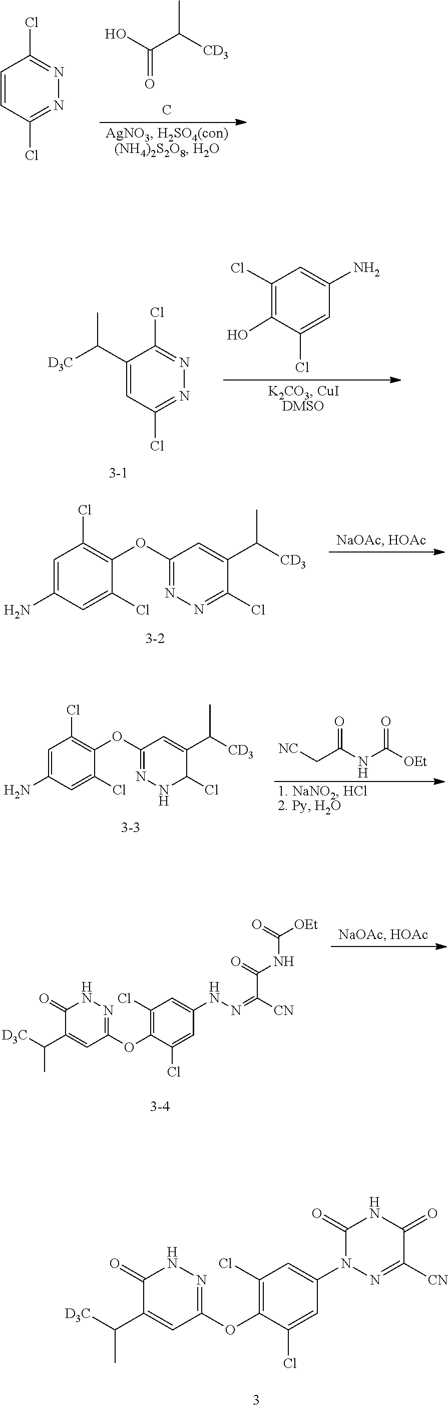

### Synthesis of 2-(trideuteromethyl)propionic acid (compound C)

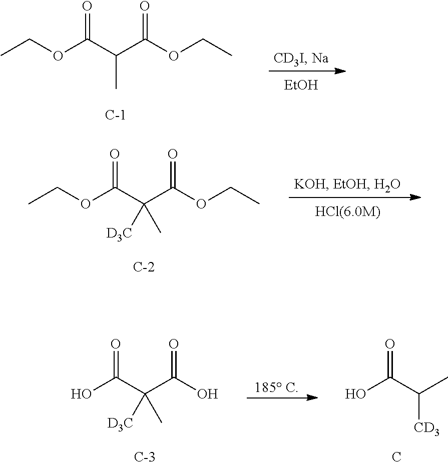

### 1) Synthesis of compound diethyl-2-deuteromethyl-2-methylmalonate

350 mL ethanol was measured and placed in a 500 mL three-necked round-bottom flask, and stirred at room temperature. Then, flaky Na (9.9 g, 430.79 mmol) was slowly added to the system in batches, and when the system was completely clear, the system was transferred to an oil bath to continue heating and stirring. When the internal temperature of the system rose to 70° C., diethyl 2-methylmalonate (20.0 g, 114.80 mmol) was added dropwise to the system. After addition, the mixture was stirred for 15 min under the same temperature, to which was added iodomethane-d₃ (18.5 g, 196.6 mmol) dropwise, and the dropping rate was controlled to keep the reflux state in the system. After addition, the system was continually allowed to stir and react in an oil bath at 90° C. After 4 h, TLC was used to monitor the complete consumption of starting materials. The oil bath was removed, and the system was cooled to room temperature, and the solvent was removed by rotary evaporation to obtain a crude product, which was separated by column chromatography to obtain diethyl 2-deuteromethyl-2-methylmalonate (15.7 g), as colorless and transparent oily liquid. Yield: 71.5%.

### (2) Synthesis of compound 2-trideuteromethyl-2-methylmalonic acid

Diethyl-2-trideuteromethyl-2-methylmalonate (10.0 g, 52.3 mmol) was weighed and moved into a 250 mL three-necked round-bottom flask, to which was added 12.5 mL ethanol, and the reaction was stirred at room temperature to dissolve and allow the solution become clear. Then, 12.5 mL aqueous KOH solution (16.8 g, 30 mmol) was added to the system. After addition, the system was moved to an oil bath at 85° C., and the reaction was continually heated and stirred. After 1 h, heating was stopped, and the system was naturally cooled to room temperature and stirred overnight. The next day, the system was placed in an ice water bath to cool down under stirring. When the internal temperature of the system was reduced to 0° C., HCl (6.0 M) solution was added dropwise to the system to adjust the pH value of the system to about 2, and the dropping rate was controlled to make the internal temperature not exceed 5° C. After that, the solvent was concentrated under reduced pressure, and then toluene was added to co-evaporate with the water in the system, that was repeated several times until the system appeared completely dry. Then, ACN (100 mL) was added to the system, and the system was placed in an oil bath at 85° C., refluxed under stirring for 15 min, and filtered while hot. The filter cake was collected, and this operation was repeated three times. The filtrate was combined, and the solvent was removed by rotary evaporation to obtain a brown solid, that was placed in a 50 mL single-necked round-bottom flask, to which was added toluene (15 mL). The mixture was stirred at room temperature to make a slurry, and after 20 min, the operation of suction filtration was performed. The filter cake was rinsed with a small amount of toluene (5 mL) several times and dried under vacuum, to obtain 2-deuteromethyl-2-methylmalonic acid as off-white solid (5.8 g). Yield 82%. MS (ESI) m/z 153.3 [M+H₂O]⁺, ¹H NMR (400 MHz, DMSO-d₆) δ 13.58 (s, 2H), 1.25 (s, 3H).

### (3) Synthesis of compound 2-trideuteromethyl-2-methylmalonic acid

Compound 2-trideuteromethyl-2-methylmalonic acid (3.1 g, 22.94 mmol) was added to a 25 mL round-bottom flask, and after heating to 180° C. and reacting for 30 min, bubbles formed, and the solid became oily. After cooling, it was distilled to obtain compound 2-trideuteromethyl propionic acid (1.1 g). Yield: 52.6%.

Using compound C and 3,6-dichloropyridazine as starting materials, compound 3a was prepared by the method similar to that in Example 1.

Step 1: Synthesis of 3,6-dichloro-4-(1,1,1-heptadeuteriopropan-2-yl)pyridazine (compound 3-1): Yield 60%, MS (ESI) m/z 194.2 [M+H]⁺.

Step 2: Synthesis of 3,5-dichloro-4-((6-chloro-5-(1,1,1-heptadeuteriopropan-2-yl)pyridazine-3-yl)oxy)aniline (compound 3-2): Yield 64%, MS (ESI) m/z 335.0 [M+H]⁺.

Step 3: Synthesis of 6-(4-amino-2,6-dichlorophenoxy)-4-(propan-2-yl-1,1,1-trideuterio) pyridazine-3(2H)-one (compound 3-3): Yield 72.5%, MS (ESI) m/z 317.1 [M+H]⁺.

Step 4: Synthesis of ethyl (2-cyano-2-(2-(3,5-dichloro-4-((6-oxo-5-(propan-2-yl-1,1,1-trideuterio)-1,6-dihydropyridazine-3-yl) oxy)phenyl)hydrono)ethyl)carbamate (compound 3-4): Yield 75%, MS (ESI) m/z 484.1 [M+H]⁺.

Step 5: Synthesis of 2-(3,5-dichloro-4-((6-oxo-5-(propan-2-yl-1,1,1-trideuterio)-1,6-dihydropyridazine-3-yl)oxy)phenyl)-3,5-dioxo-2,3,4,5-tetrahydro-1,2,4-triazine-6-carbonitrile (compound 3): Yield: 41.4%. MS (ESI) m/z 438.1 [M+H]⁺; ¹H NMR (400 MHz, DMSO-d₆) δ 13.29 (s, 1H), 12.25 (s, 1H), 7.79 (s, 2H), 7.45 (s, 1H), 3.04 (q, J=6.5 Hz, 1H), 1.21-1.19 (d, 3H).

### Example 4 Synthesis of 2-(3,5-dichloro-4-((4-deuterio-5-(heptadeuteroisopropyl)-6-oxo-1,6-dihydro-pyridazin3-yl)oxy)phenyl)-3,5-dioxo-2,3,4,5-tetrahydro-1,2,4-triazine-6-carbonitrile (10)

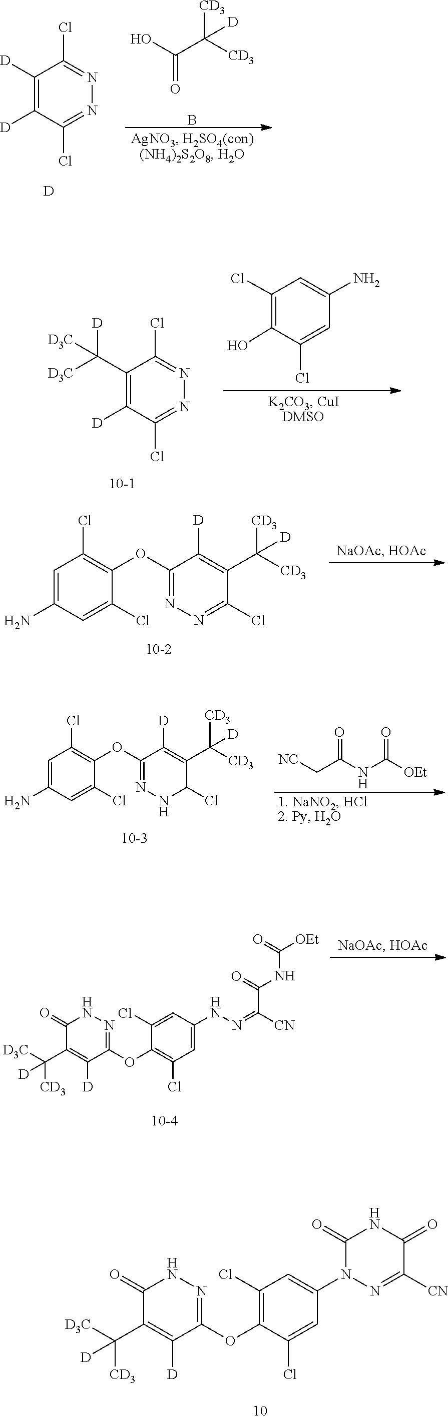

### Synthesis of 4,5-dideuterio-3,6-dichloropyridazine (compound D)

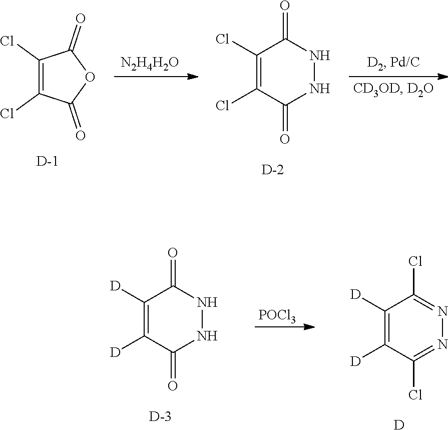

### (1) Synthesis of 4,5-dichloro-maleic hydrazide (D-2)

Compound 2,3-dichloromaleic anhydride (8.35 g, 50 mmol) was weighed and added to a 100 ml round-bottom flask, to which were successively added 40 ml water and hydrazine hydrate (2.5 g, 50 mmol), and the mixture was heated to reflux and reacted for 4 h. Then, the system was cooled to room temperature and kept in an ice-water bath for 30 min. The mixture was filtered, the filter cake was rinsed with 100 ml water and dried, to obtain 4,5-dichloromaleic hydrazide (D-2, 5.0 g), yield: 55.3%, MS (ESI) m/z 181.0 [M+H]⁺.

### (2) Synthesis of 4,5-dideuterio-maleic hydrazide (D-3)

Method 1: 4,5-Dichloromaleic hydrazide (2.0 g, 11.05 mmol) was weighed and added in a 100 ml single-necked round-bottom flask, to which were subsequently added 50 ml methanol-d₄, 10 ml heavy water, 200 mg Pd/C, and then the flask was purged with deuterium gas 3 times. The mixture was allowed to react at room temperature for 40 h, and filtered. The filtrate was concentrated to dryness under reduced pressure, to which was added 6 ml methanol for slurrying, followed by filtering. The filter cake was dried to obtain compound 4,5-dideuteriomaleic hydrazide (1.0 g), yield: 79%, MS (ESI) m/z 115.2 [M+H]⁺. ¹³C NMR (101 MHz, DMSO-d₆) δ 156.76, 130.50.

Method 2: Maleic hydrazide (5.6 g, 50 mmol) was added to a round-bottom flask, to which were successively added 80 ml heavy water and 500 mg Pd/C, and the flask was purged with hydrogen three times. The reaction was heated and refluxed for 72 h in a hydrogen atmosphere, and then cooled to room temperature. The mixture was filtered, and the filter cake was added to a round-bottom flask, and the above process was repeated. After the reaction was completed, the mixture was filtered, and 100 ml methanol was added to the filter cake. The mixture was refluxed for 30 min, filtered, and the filtrate was concentrated to dryness under reduced pressure, to obtain 4,5-dideuterio-maleic hydrazide (2.5 g), with a yield of 43.87%.

### (3) Synthesis of 4,5-dideuterio-3,6-dichloropyridazine (D)

4,5-Dideuteriomaleic hydrazide (1.0 g, 8.74 mmol) was weighed and placed in a 100 ml round-bottom flask, to which was added 15 ml phosphorus oxychloride, and the mixture was refluxed at 115° C. for 4 h, concentrated under reduced pressure to dry, and then cooled in an ice-water bath. 20 ml ice water was added, and pH was adjusted to 9.0 with ammonia water, to which was added 30 ml dichloromethane for extraction. The aqueous layer was further extracted with 20 ml dichloromethane once, and the organic layer was combined, and washed with water and saturated brine, respectively, then dried on anhydrous sodium sulfate. The solvent was concentrated to dryness under reduced pressure, to obtain 1.2 g compound 4,5-dideuterio-3,6-dichloropyridazine, with a yield of 90.9%. ¹³C NMR (101 MHz, DMSO-d₆) δ 156.3, 131.9 (t, J=27 Hz). MS (ESI) m/z 151 [M+H]⁺.

Using compounds D and B as starting materials, compound 10 was prepared by a method similar to Example 1.

### Step 1: Synthesis of 3,6-dichloro-4-deuterio-5-(heptadeuteriopropan-2-yl)pyridazine (compound 10-1)

Compound b (377.7 mg, 3.97 mmol) was weighed and placed in a 100 mL three-necked round-bottom flask, to which was added 10 mL water, and stirred at room temperature to dissolve B and allow the solution become clear. Then, 3,6-dichloro-4,5-dideuteriopyridazine (599.3 mg, 3.97 mmol) was added to the system, and the reaction was stirred at room temperature. Then, silver nitrate (674.3 mg, 3.97 mmol) was added to the system, after which the system was transferred to an oil bath, heated and stirred for reaction. When the internal temperature of the system rose to 50° C., to which was dropwise added concentrated sulfuric acid (1 mL). After addition, the system was stirred at the temperature of 50° C. for 10 min. Then, when the internal temperature of the system rose to 60° C., 2 mL aqueous solution of ammonium persulfate (2.7 g, 11.91 mmol) was added dropwise to the system. When the internal temperature rose to 70° C., the system was allowed to react and stirred at this temperature for 30 minutes. The heating was stopped, and the system was naturally cooled to room temperature. Then, the system was transferred to an ice water bath to cool down under stirring. After 15 min, NaOH (6.0 M) solution was added dropwise to the system to adjust pH to about 8. Ethyl acetate (20 mL) was added to the system, and the mixture was stirred vigorously and stood for separating the layers. The aqueous phase was back-extracted with ethyl acetate (15 mL×3), the organic phase was combined, and then successively washed with water (10 mL×3) and saturated brine (15 mL), followed by drying over anhydrous sodium sulfate. The solvent was concentrated under reduced pressure to obtain a crude product, which was separated by column chromatography to obtain compound 10-1 (520 mg) as white solid, with a yield of 65.8%. MS (ESI) m/z 199.1 [M+H]⁺.

### Step 2: Synthesis of 3,5-dichloro-4-((6-chloro-4-deuterio-5-(heptadeuteriopropan-2-yl) pyridazine-3-yl)oxy)aniline (compound 10-2)

10-1 (520 mg, 2.61 mmol) was weighed and placed in a 25 mL three-necked round-bottom flask, to which was added 5 mL dimethyl sulfoxide, and the mixture was stirred at room temperature to dissolve and become clear, followed by successive addition of 4-amino-2,6-dichlorophenol (464.6 mg, 2.61 mmol), anhydrous potassium carbonate (1.4 g, 10.44 mmol), and cuprous iodide (299 mg, 1.57 mmol). After addition, the operation of purging argon was performed and repeated ten times to ensure an inert gas atmosphere in the system. Then, the system was transferred to an oil bath at 90° C., and the reaction was heated and stirred overnight. After 24 h, the disappearance of starting materials was monitored. Then, the heating was stopped, and the system was allowed to naturally cool to room temperature. To the system, were added ethyl acetate (20 mL) and water (20 mL), and the reaction was stirred vigorously, and then stood to separate the layers. The aqueous phase was back-extracted with ethyl acetate (20 mL×3), and the organic phase was combined and successively washed with water (20 mL×3) and saturated brine (20 mL), then dried on anhydrous sodium sulfate. The solvent was concentrated under reduced pressure to obtain a crude product, which was then separated by column chromatography to obtain compound 10-2 as white solid (668 mg), with a yield of 75.1%. MS (ESI) m/z 340.1 [M+H]⁺. ¹H NMR (400 MHz, DMSO-d₆) δ 6.71 (s, 2H), 5.67 (s, 2H).

### Step 3: Synthesis of 6-(4-amino-2,6-dichlorophenoxy)-5-deuterio-4-(heptadeuteriopropan-2-yl) pyridazine-3(2H)-one (compound 10-3)

Compound 10-2 (668 g, 1.96 mmol) was weighed and placed in a 50 mL three-necked round-bottom flask, to which was added glacial acetic acid (20 mL), and the mixture was stirred at room temperature. Then, anhydrous sodium acetate (562.7 mg, 6.86 mmol) was added to the system. After addition, the system was placed in an oil bath at 105° C., stirred, and refluxed for reaction. After 22 h, heating was stopped, and the system was allowed to naturally cool to room temperature. The solvent was removed by rotary evaporation, water (100 mL) was added to the system, and the system was then transferred to an ice-water bath to cool down under stirring. When the internal temperature of the system dropped to 5° C., sodium hydroxide (1.0 M) solution was added dropwise to adjust the pH value to about 9. Then, to the system was added ethyl acetate (100 mL), and the mixture was stirred vigorously, and then stood to separate the layers. The aqueous phase was back-extracted with ethyl acetate (50 mL×2), and the organic phase was combined and washed once with water (30 mL) and saturated brine (30 mL), respectively, followed by drying over anhydrous sodium sulfate and concentrating under reduced pressure, to obtain a pale yellow solid. Methanol (20 mL) and NaOH (1.0 M) solution (20 mL) were sequentially added to a 100 mL three-necked round-bottom flask containing the solid. After addition, the system was transferred to an oil bath at 105° C. and refluxed for reaction. After 11 h, heating was stopped, the oil bath was removed, and the system was allowed to return to room temperature. The solvent was removed by rotary evaporation, and then ethyl acetate (160 mL) and water (100 mL) were added. The mixture was stirred vigorously, and then stood to separate the layers. The aqueous layer was back-extracted with ethyl acetate (25 mL×2), and the organic layers were combined, then washed successively with water (20 mL×2) and saturated brine (20 mL), followed by drying with anhydrous sodium sulfate and removing the solvent by rotary evaporation, to obtain a crude product, which was then separated by column chromatography to obtain compound 10-3 (416 mg) as solid, with a yield of 65.8%. MS (ESI) m/z 322.1 [M+H]⁺. ¹H NMR (400 MHz, DMSO-d₆) δ 12.10 (s, 1H), 6.66 (s, 2H), 5.60 (s, 2H).

### Step 4: Synthesis of ethyl (2-cyano-2-(2-(3,5-dichloro-4-((4-deuterio-5-heptadeuteroisopropyl-6-oxo-1,6-dihydropyridazine-3-yl)oxy)phenyl)hydrono)acetyl)carbamate (compound 10-4)

Compound 10-3 (300 mg, 0.93 mmol) was weighed and moved to a 50 mL single-necked round-bottom flask, to which was added water (12.5 mL), and the mixture was stirred at room temperature. Then, concentrated hydrochloric acid (6.5 mL) was added to the system. After addition, the system was placed in an ice water bath to cool down under stirring. When the internal temperature dropped to 0° C., 1.0 mL aqueous solution of sodium nitrite (80 mg, 1.16 mmol) was added dropwise to the system. Then, the system was continually stirred and reacted 30 min at the temperature of 0° C. In addition, N-cyanoacetylurethane (159.2 mg, 1.02 mmol) was weighed and placed in a 50 mL single-necked round-bottom flask, to which were added water (9.4 mL) and pyridine (2.8 mL), and the mixture was stirred at room temperature to dissolve and make it clear. Then, the system was moved to an ice-water bath to continue cooling and stirring 30 min. The diazotization reaction solution was slowly added dropwise to the system containing N-cyanoacetylurethane, and the dropping rate was controlled so that the internal temperature of the system did not exceed 5° C. After addition, the system was still left in the ice-water bath to react under stirring. After 1 h, TLC was used to monitor the completion of the reaction. The system was subjected to the operation of suction filtration, the filter cake was rinsed several times with a small amount of water, and then rinsed several times with n-hexane. After drying, an orange solid 10-4 (124 mg) was obtained. Without further purification, it was directly used in the next reaction. Yield: 92.1%. MS (ESI) m/z 489.1 [M+H]⁺.

### Step 5: Synthesis of 2-(3,5-dichloro-4-((4-deuterio-5-(heptadeuteroisopropyl)-6-oxo-1,6-dihydro-pyridazin3-yl)oxy)phenyl)-3,5-dioxo-2,3,4,5-tetrahydro-1,2,4-triazine-6-carbonitrile (compound 10)

Compound 10-4 (300 mg, 0.61 mmol) was weighed and placed in a 25 mL single-necked round-bottom flask, to which was added glacial acetic acid (8 mL), and the mixture was stirred at room temperature. Then, anhydrous sodium acetate (250.1 mg, 3.05 mmol) was added to the system. After that, the system was moved to an oil bath at 120° C. to increase the temperature under stirring for reaction. After 2 h, TLC was used to monitor the consumption of starting materials. Heating was stopped, the system was cooled to room temperature, and then placed in an ice-water bath to continue cooling and stirring. When the internal temperature dropped to 5° C., ice water was added to the system and then stirred vigorously for 30 min. Subsequently, it was subjected to the operation of suction filtration, the filter cake was rinsed with a small amount of water for several times, and then dissolved in ethyl acetate, followed by drying over anhydrous sodium sulfate and removing the solvent by rotary evaporation to obtain a crude product, which was separated and purified by Pre-TLC to obtain compound 10 as light orange solid (190 mg), with a yield of 69.9%. MS (ESI) m/z 443.0 [M+H]⁺. ¹H NMR (400 MHz, DMSO-d₆) δ 12.22 (br, 1H), 7.78 (s, 2H).

### Example 5 Synthesis of 2-(3,5-dichloro-4-((4-deuterio-5-(1, 1, 1, 3, 3, 3-hexadeuteropropan-2-yl)-6-oxo-1,6-dihydro-pyridazin3-yl)oxy)phenyl)-3,5-dioxo-2,3,4,5-tetrahydro-1,2,4-triazine-6-carbonitrile (11)

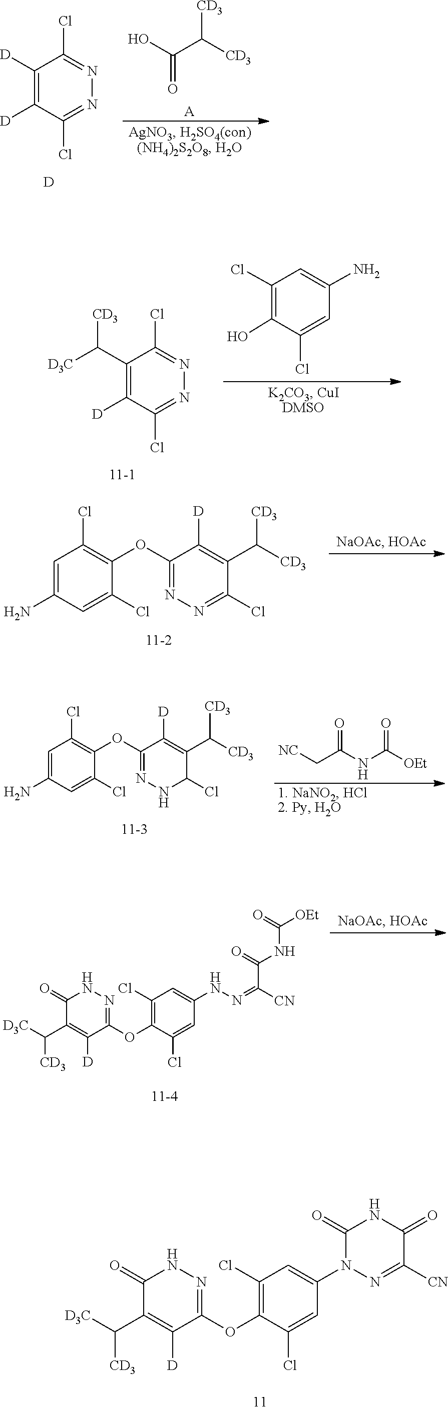

Using compounds D and A as starting materials, compound 11 was prepared by a method similar to the synthesis of compound 10.

### Step 1: Synthesis of 3,6-dichloro-4-deuterio-5-(1,1,1,3,3,3-hexadeuteropropan-2-yl)pyridazine (compound 11-1)

Compound 3,6-dichloro-4,5-dideuteriopyridazine (604 mg, 4.0 mmol) was dissolved in 10 ml water, to which was added compound C (372 mg, 4.0 mmol), and then AgNO₃ (680 mg, 4 mmol) was added under stirring. The temperature rose to 50° C., and then concentrated sulfuric acid (1 mL) was added dropwise. After addition, the temperature rose to 60° C. and reacted 10 min, and then 6 mL aqueous solution of ammonium persulfate (2.74 g, 12 mmol) was added dropwise to the system. After addition, the temperature rose to 70° C. and reacted for 30 min, until TLC was used to monitor the disappearance of starting materials. The heating was stopped, and the system was cooled down in an ice-water bath. Then, 6 N NaOH aqueous solution was used to adjust pH to 8.0, and extracted with ethyl acetate (30 mL). The organic layer was sequentially washed with water and saturated brine, and then dried on anhydrous sodium sulfate and concentrated to dry under reduced pressure. The residue was separated and purified by column chromatography (petroleum ether/ethyl acetate=10:1), to provide compound 11-1 (500 mg), with a yield of 63.1%, MS (ESI) m/z 198.1 [M+H]⁺.

### Step 2: Synthesis of 3,5-dichloro-4-((6-chloro-5-(1,1,1,3,3,3-hexadeuteropropan-2-yl) pyridazine-3-yl-4-deuterio)oxy)aniline (compound 11-2)

Compound 11-1 (500 mg, 2.52 mmol) was dissolved in 10 ml DMSO, to which was added compound 4-amino-2,6-dichlorophenol (454 mg, 2.52 mmol), then K₂CO₃ (1.41 g, 10.2 mmol) was added under stirring, followed by addition of CuI (292 mg, 1.53 mmol). The system was purged with Ar three times, and then heated 90° C. and reacted overnight. The system was cooled down, to which was added water, and then extracted with ethyl acetate. The organic layer was respectively washed with water and saturated brine, dried over anhydrous sodium sulfate, and concentrated to dry under reduced pressure. The residue was separated and purified by column chromatography (petroleum ether/ethyl acetate=5:1), to provide compound 11-2 (452 mg), with a yield of 52.8%, MS (ESI) m/z 339.2 [M+H]⁺; ¹H NMR (400 MHz, DMSO-d₆) δ 6.72 (d, J=2.7 Hz, 2H), 5.69 (s, 2H), 3.12 (s, 1H).

### Step 3: Synthesis of 6-(4-amino-2,6-dichlorophenoxy)-4-(1,1,1,3,3,3-hexadeuteropropan-2-yl) pyridazine-5-deuterio-3(2H)-one (compound 11-3)

Compound 11-2 (440 mg, 1.30 mmol) was dissolved in 5 ml acetic acid, to which was added sodium acetate (374 mg, 4.60 mmol), and the mixture was refluxed at 105° C. overnight, then concentrated to dry under reduced pressure. The residue was adjusted to pH=9 using 6 N NaOH solution, and then extracted with 20 ml ethyl acetate. The water layer was further back-extracted with 20 ml ethyl acetate, and the organic layer was combined and sequentially washed with water and saturated brine, respectively, followed by drying over anhydrous sodium sulfate, and concentrating to dry under reduced pressure. To the residue, was added 10 ml methanol, followed by addition of 10 ml 1N NaOH aqueous solution. The mixture reacted 6 h at 105° C., and was cooled down and concentrated under reduced pressure to remove most of methanol. To the residue, was added 20 ml ethyl acetate to extract. The organic layer was washed with water and saturated brine, dried over anhydrous sodium sulfate, and concentrated to dry under reduced pressure. The residue was separated and purified by column chromatography (petroleum ether/ethyl acetate=3:2), to provide compound 11-3 (202 mg), with a yield of 48.3%, MS (ESI) m/z 321.2 [M+H]⁺; ¹H NMR (400 MHz, DMSO-d₆) δ 12.10 (s, 1H), 6.67 (d, J=4.3 Hz, 2H), 5.60 (s, 2H), 2.98 (s, 1H).

### Step 4: Synthesis of ethyl (2-cyano-2-(2-(3,5-dichloro-4-((4-deuterio-5-(1,1,1,3,3,3-hexadeuteropropan-2-yl)-6-oxo-1,6-dihydropyridazin-3-yl)oxy)phenyl)hydrono)ethyl)carbamate (compound 11-4)

Compound 11-3 (195 mg, 0.61 mmol) was added to 10 ml water, to which was added 4.6 ml concentrated hydrochloric acid under stirring, and then transferred to an ice-water bath, followed by addition of sodium nitrite (52 mg, 0.76 mmol), and the mixture was stirred for 30 min at this constant temperature. N-cyanoacetylurethane (105 mg, 0.67 mmol) was added to a round-bottom flask, to which were successively added 16 ml water and 4.6 ml pyridine, and then placed in an ice-water bath 30 min. The reaction solution from the previous step was added dropwise to the reaction system. After addition, the mixture was stirred for 30 min in an ice-water bath, filtered, and the filter cake was rinsed with 100 ml water and dried to obtain a purple-red solid compound 11-4 (265 mg), with a yield of 89%. MS (ESI) m/z 488.1 [M+H]⁺.

### Step 5: Synthesis of 2-(3,5-dichloro-4-((4-deuterio-5-(1,1,1,3,3,3-hexadeuteropropan-2-yl)-6-oxo-1,6-dihydro-pyridazin-3-yl)oxy)phenyl)-3,5-dioxo-2,3,4,5-tetrahydro-1,2,4-triazine-6-carbonitrile ( 11)

Compound 11-4 (265 mg, 0.54 mmol) was dissolved in 4 ml acetic acid, sodium acetate (222 mg, 2.7 mmol) was added, and the reaction was refluxed at 125° C. for 2 h. After cooling to room temperature, 50 ml ice water was added, and then the mixture was cooled in an ice-water bath and stirred for 30 min, and filtered. The filter cake was rinsed with 80 ml water, and dried in an oven. The filter cake was dissolved in 50 ml methanol, to which was added 100 mg activated carbon, and the mixture was refluxed at 80° C. for 30 min. The mixture was filtered while heating, and the filtrate was concentrated to dryness under reduced pressure to obtain compound 11 (195 mg) as white solid, with a yield of 81.7%. MS (ESI) m/z 442.0 [M+H]⁺; ¹H NMR (400 MHz, DMSO-d₆) δ 13.30 (br, 1H), 12.25 (br, 1H), 7.79 (s, 2H), 3.01 (s, 1H).

Using the corresponding starting materials and the preparative methods similar to those described in Examples 1-5, compounds 4-9 and 12 can be prepared: using compound C3 as starting material as well as the method similar to the synthesis of heptadeuterioisobutyric acid compound B, 2,3,3,35-tetradeuterio-2-methylpropionic acid can be prepared. Using it as a starting material, compound 4 can be prepared by a method similar to that in Example 1.

Using 2,2-dimethylmalonic acid as a starting material, together with a method similar to the synthesis of compound B, 2-deuterio-2-methylpropionic acid can be prepared. Using it as a raw material, compound 5 can be prepared by a method similar to that in Example 1.

Using isobutyric acid and 4,5-dideuterio-3,6-dichloropyridazine as starting materials, compound 6 can be prepared by a method similar to that in Example 4.

Using isobutyric acid, 4,5-dideuterio-3,6-dichloropyridazine and 4-amino-2,6-dichloro-3,5-dideuteriophenol (by the method similar to that reported in Organic Letters, 2008, 10, 4351.) as starting materials, compound 7 can be prepared by a method similar to that in Example 4.

Using hexadeuterioisobutyric acid A and 4-amino-2,6-dichloro-3,5-dideuteriophenol as starting materials, compound 8 can be prepared by a method similar to that in Example 1.

Using heptadeuterioisobutyric acid B and 4-amino-2,6-dichloro-3,5-dideuteriophenol as starting materials, compound 9 can be prepared by a method similar to that in Example 1.

Using heptadeuterioisobutyric acid B, 4,5-dideuterio-3,6-dichloropyridazine, and 4-amino-2,6-dichloro-3,5-dideuteriophenol as starting materials, compound 12 can be prepared by a method similar to that in Example 4.

In the following, the beneficial effects of the present invention were illustrated by experimental examples.

### Experimental Example 1 the Agonistic Activity Test of the Compound of the Present Invention on THR−

A method similar to the literature (J. Med. Chem. 2014, 57, 3912.) was used to determine the agonistic activity of the compound on THR−:

100× reference compound or compound was prepared with DMSO, and 1:3 equal ratio dilution was carried out. 100× gradient dilution reference compound or compound was diluted to 4× with 1× reaction buffer and added to the experimental plate. 4× TRα-LBD or TRP-LBD, as well as 4× RXRα mixed solution was prepared with 1× reaction buffer, and added to the experimental plate. A mixed solution of 2× biotin-SRC2-2, 2× Eu-anti-GST, and 2× streptavidin-d2 was prepared with 1× reaction buffer, and added to the experimental plate. The plate was centrifuged at 1000 rpm for 1 min, and incubated for 4 h at room temperature and protected from light. The fluorescence signal values were read at 665 nm and 615 nm on EnVision 2104 plate reader, and Ratio_{665 nm/615 nm} was calculated. The experimental results are shown in Table 1.

TABLE 1
The agonistic activities of compounds on THR-β.

| Compounds | EC₅₀ (nM) |
|---|---|
| MGL-3196 | 240 |
| 1 | 244 |
| 2 | 97 |
| 3 | 97 |
| 10 | 168 |
| 11 | 121 |

Experiments showed that the compounds of the present invention had good agonistic activity on THR-, particularly compounds 2, 3, 10, and 11, whose agonistic activities on THR- were significantly better than the non-deuterated control compound MGL-3196.

### Experimental Example 2 Experiment on the Metabolic Stability of Compounds of the Present Invention in Liver Microsomes

1. Preparation of NADPH solution (5 mM): An appropriate amount of NADPH standard was weighed and dissolved in phosphate buffer (1×PBS), that was mixed well and placed in an ice bath for use; Preparation of test compound solution: an appropriate amount of test substances were respectively weighed, and a solution with a suitable concentration was prepared with DMSO, to obtain stock solution I; an suitable amount of stock solution I was further diluted to 5 mM with 1×PBS to obtain stock solution II, that was stored in a refrigerator at 4° C. for later use; Preparation of liver microsome solution: the original solutions of rat, mouse, and human liver microsomes were respectively diluted to 0.833 mg/ml with PBS. 2. 60 μl of liver microsome solution was transferred to a 1 ml 96-well plate, to which was added 20 μl of the test drug solution (or probe substrate testosterone solution), and then pre-incubated for 5 min in a 37° C. thermomixer. 20 μl NADPH solution was added to start the reaction, and the contents of test drug, microsomal protein and NADPH in the reaction system were 1 μM, 0.5 mg/ml and 1 mM, respectively, and the content of DMSO in the system was not higher than 0.2%. Then, at 0, 15, 30, 45, 60 min (duplicated wells for each time point), 300 μl acetonitrile was added to stop the reaction (including the internal standard compound suitably selected).

3. 20 μl NADPH was replaced with an equal volume of 1×PBS in the control group, and the contents of test drug and microsomal protein in the reaction system were 1 μM and 0.5 mg/ml, respectively, while the content of DMSO in the system was not higher than 0.2%. Respectively at 0 min and 60 min (duplicated wells at each time point), 300 μl acetonitrile was added to stop the reaction (including the internal standard compound suitably selected).

4. After finishing the incubation time, the samples obtained by terminating the reaction were mixed, and centrifuged at 3200 rpm for 10 min, then the supernatant was collected for LC/MS/MS analysis.

5. Data analysis: The peak area was determined from the extracted ion chromatogram. The slope value k was determined by linear regression of the natural logarithm obtained from the curve that was resulted from the remaining percentage of the parent drug vs the incubation time.

In vitro half-life (in vitro t_{1/2}) was determined by the slope value: in vitro t_{1/2}=−(0.693/k) In vitro CLᵢₙₜ (in the unit of L/min/mg) was converted from the in vitro half-life t_{1/2} (minutes) using the following equation (the average of repeated determinations):

$$in vitro {CL}_{int} = \frac{0.693}{{t}_{1 / 2}} \times \frac{volume of incubation ( µL )}{amount of proteins ( mg )}$$

Scale up CLᵢₙₜ (in the unit of mL/min/kg) was converted from in vitro t_{1/2} (minutes) by using the following formula (the average of repeated determinations):

$$Scale up {CL}_{int} - \frac{0.693}{{t}_{1 / 2}} \times \frac{volume of incubation ( µL )}{amount of proteins ( mg )} \times Scaling Factor$$

The experimental results of the metabolic stability in liver microsomes of mouse, rat, and human are shown in Table 2:

TABLE 2
The experimental results of the metabolic stability
in liver microsomes of mouse and human.

| | Mouse liver | Human liver |
|---|---|---|
| | microsomes | microsomes |
| | Half-life t_{1/2} (min) | Half-life t_{1/2} (min) |
| MGL-3196 | 486 | 478 |
| Compound 1 | 527 | 415 |
| Compound 2 | ∞ | 1270 |
| Compound 10 | 882 | 641 |
| Compound 11 | 458 | 1176 |

As shown in the above table, the half-life of the compound according to the present invention in mouse and human liver microsomes was longer than that of the non-deuterated compound MGL3196, especially compound 2, compound 10, and compound 11, indicating that the compound of the present invention was more metabolically stable than non-deuterated compound MGL3196.

This suggested the compound of the present invention may have better pharmacokinetics, better safety and effectiveness.

### Experimental Example 3 Metabolic Stability of the Compound According to the Present Invention in Human CYP2C8 Metabolic Enzymes

1) Na₂HPO₄, KH₂PO₄, and pure water were used to prepare phosphate buffer (100 mM, pH 7.4);

2) Test compound and reference compound were dissolved in acetonitrile to obtain a working solution (200 M);

3) NADPH solution (10 mM) was prepared with NADPH and phosphate buffer (100 mM, pH 7.4);

4) The stock solution of recombinant CYP2C8 enzyme was diluted to 100 μM with phosphate buffer (100 mM, pH 7.4);

5) The working solution of test compound or reference compound Amitriptyline was added to CYP2C8 solution, and the concentration of compound was 2 M, to which was added NADPH solution (10 mM) to initiate a metabolic reaction. 30 L incubation solution of metabolic reaction was collected at 0, 5, 10, 15, 25 minutes, and transferred to a quenching plate to quench with 120 L acetonitrile (including internal standard);

6) The sample was centrifuged at 4° C. for 60 min to precipitate the protein. The supernatant was collected and transferred to a 96-well plate, then diluted one times with pure water, and subjected to LC-MS/MS analysis;

7) The data analysis and calculation were similar to experimental example 2. The results obtained are shown in Table 3.

TABLE 3
The metabolic stability of the compound according
to the present invention vs CYP2C8.

| | | | Percentage | | |
|---|---|---|---|---|---|
| | | Test | of residual | Half- | Clearance |
| | CYP | concen- | substances | life | rate CLint |
| | sub- | tration | after 25 | T_{1/2} | (μL/min/pmol |
| Compound | type | (M) | minutes (%) | (min) | CYP450) |
| Amitriptyline | 2C8 | 2 | 18.81 | 10.6 | 0.65 |
| MGL-3196 | 2C8 | 2 | 72.96 | 62.16 | 0.11 |
| Compound 1 | 2C8 | 2 | 77.70 | 72.61 | 0.10 |
| Compound 2 | 2C8 | 2 | 80.87 | 90.75 | 0.08 |
| Compound 10 | 2C8 | 2 | 81.60 | 96.06 | 0.07 |
| Compound 11 | 2C8 | 2 | 85.57 | 110.03 | 0.06 |

CYP2C8 was the main human metabolic enzyme of MGL-3196. The metabolic stability of CYP2C8 could better predict the metabolic stability of the compound according to the present invention in human body. Experiments showed that under the action of CYP2C8, the compound of the present invention had a longer half-life and lower clearance rate than MGL-3196, especially compounds 2, 10, and 11. Therefore, for CYP2C8, the compound of the present invention had better metabolic stability and was expected to have better human pharmacokinetics.

### Experimental Example 4 Pharmacokinetics of the Compounds According to the Present Invention in Mice

1) Experimental Materials and Instruments:
N,N-Dimethylacetamide (DMA), manufacturer: Sigma; Polyethylene glycol 400 (PEG400), manufacturer: Chengdu Kelon Chemical Reagent Factory; Hydroxypropyl β-cyclodextrin (HP-3-CD), manufacturer: Shanghai Dibai Chemical Technology Co., Ltd.;
HPC LF, manufacturer: Chengdu Yuannuo Tiancheng Technology Co., Ltd.;
Heparin sodium, manufacturer: Chengdu Kelon Chemical Reagent Factory;
Electronic analytical balance, model: SECURA225D-1CN; manufacturer: Sartorius Group, Germany;
Ultrasonic Cleaner, Model: AS10200; Manufacturer: Tianjin Automatic Science Instrument Co., Ltd.;
Pure water system, model: PURELAB Classic; manufacturer: ELGA Instrument Co., Ltd., Britain;
Vortex meter, model: VORTEXI; manufacturer: IKA Group, German;
High-speed refrigerated centrifuge, model: 21R; manufacturer: ThermoFisher Scientific (China) Co., Ltd.;
Electronic balance, model: XY1000-2C; manufacturer: Changzhou Xing Yun Electronic Equipment Co., Ltd.;
HPLC system, model: LC-20AD; manufacturer: SHIMADZU Instrument Company, Japan;
API4000 triple quadrupole mass spectrometer, manufacturer: Applied Biosystem, USA;
LC-20AD HPLC system (SHIMADZU (Shimadzu) Company, Japan);
PhenixWinnolin Pharmacokinetic software (Version 6.3, Certara, USA);
Analytical balance (Sartorius, SECURA225D-1CN);
Experimental animals: ICR mice (Chengdu Dossy Experimental Animal Co., Ltd.)

2) Experimental Methods and Results

Preparation of Samples to be Tested:

Group IV: The sample to be tested (1.15 mg) was accurately weighed, to which was first added 0.228 ml DMA to dissolve it, then were successively added 1.139 ml PEG400 and 5.012 ml of 0.1M phosphate buffer. Finally 40% HP-B-CD was added to a final volume of 11.39 ml. The mixture was sonicated, vortexed, and mixed well, to prepare a transparent and clear solution of 0.1 mg/ml. PO group: The sample to be tested (5.06 mg) was accurately weighed, and then 2% HPC LF (containing 0.1% Tween-80) was added to a final volume of 20.04 ml. The mixture was sonicated, vortexed, and mixed, to make a uniform suspension solution of 0.25 mg/ml.

Experimental Procedure:

Nine healthy adult ICR mice (three animals for each time point); after fasting overnight (free drinking water), they were administrated by tail vein and gavage, respectively; For the iv group, 5 min, 15 min, 0.5 h, 1 h, 2 h, 4 h, 8 h, 12 h, 24 h after administration, 0.1 ml blood was collected from the submandibular vein, centrifuged at 4° C. for 5 min to separate the plasma, and then it was stored at −20° C. for testing. In the po. group, 0.1 ml blood was collected from the submandibular vein prior to administration and 0.5 h, 1 h, 2 h, 4 h, 6 h, 8 h, 12 h, 24 h after administration, and the processing procedure was the same as that of the intravenous administration group. LC/MS/MS method was established to determine the plasma concentration of the original drug and draw the plasma concentration-time curve. WinNonlin 6.3 software was used to calculate the main pharmacokinetic parameters.

TABLE 4
Pharmacokinetic experiment in mice (po. 5 mpk).

| | | Peak | Exposure | |
|---|---|---|---|---|
| | Peak time | concentration | dose AUC | Half life |
| Example No. | Tₘₐₓ (h) | Cₘₐₓ (g/mL) | (μg*h/mL) | T_{1/2} (h) |
| MGL-3196 | 2 | 1.37 | 9.96 | 2.81 |
| Compound 1 | 2 | 3.2 | 18.5 | 3.19 |
| Compound 2 | 4 | 2.3 | 21.3 | 2.7 |
| Compound 3 | 4 | 1.9 | 23.1 | 3 |
| Compound 10 | 4 | 7.4 | 45.9 | 3.47 |
| Compound 11 | 4 | 6.5 | 44.1 | 4.7 |

The results of pharmacokinetic experiments in mice showed that the compounds of the present invention, especially compounds 10 and 11, had higher plasma concentration, higher exposure dose, longer half-life and better pharmacokinetic performance than MGL-3196.

In summary, compared with the non-deuterated control compound MGL3196, the compound provided in the present invention or an optical isomer, a pharmaceutically acceptable salt, a prodrug, a hydrate or a solvate thereof had better agonistic effect on thyroid hormone receptor R (THR-0), longer half-life, lower clearance rate, better metabolic stability and pharmacokinetic properties. The application prospects in the preparation of THR-0 agonists and drugs for treatment of dyslipidemia, hypercholesterolemia, non-alcoholic steatohepatitis (NASH), and non-alcoholic fatty liver disease (NAFLD) were excellent.

1. The compound of formula (I) or an optical isomer, a pharmaceutically acceptable salt, a prodrug, a hydrate or a solvate thereof: 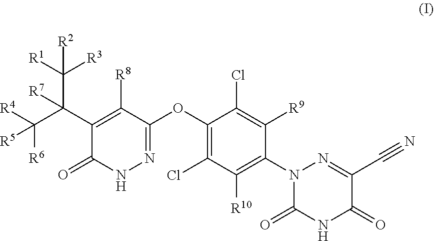
Wherein, R¹-R¹⁰ are each independently selected from H and D, and not all H.

2. The compound according to claim 1 or an optical isomer, a pharmaceutically acceptable salt, a prodrug, a hydrate or a solvate thereof, characterized in that said compound has a structure of formula (II): 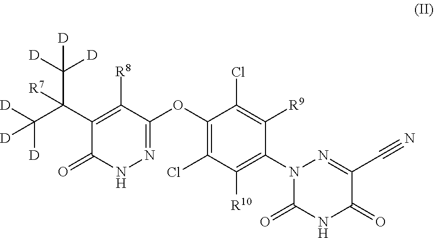
Wherein, R⁷-R¹⁰ are each independently selected from H and D.

3. The compound according to claim 1 or an optical isomer, a pharmaceutically acceptable salt, a prodrug, a hydrate or a solvate thereof, characterized in that said compound has a structure of formula (III): 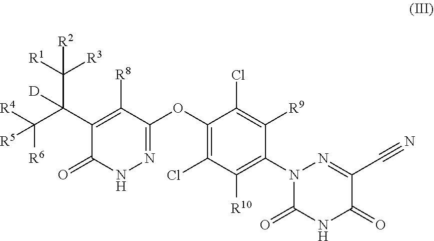
Wherein, R¹-R⁶ and R⁸-R¹⁰ are each independently selected from H and D.

4. The compound according to claim 1 or an optical isomer, a pharmaceutically acceptable salt, a prodrug, a hydrate or a solvate thereof, characterized in that said compound has a structure of formula (IV):
Wherein, R⁸-R¹⁰ are each independently selected from H and D.

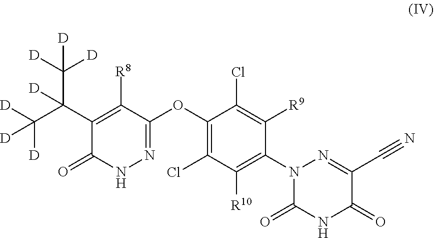

5. The compound according to claim 1 or an optical isomer, a pharmaceutically acceptable salt, a prodrug, a hydrate or a solvate thereof, characterized in that said compound has a structure of formula (V): 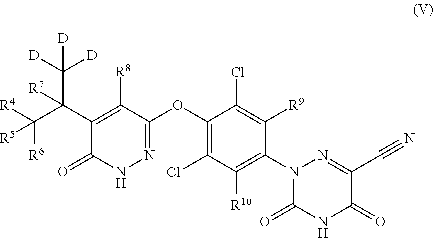
Wherein, R⁴-R¹⁰ are each independently selected from H and D.

6. The compound according to claim 1 or an optical isomer, a pharmaceutically acceptable salt, a prodrug, a hydrate or a solvate thereof, characterized in that said compound has a structure of formula (VI): 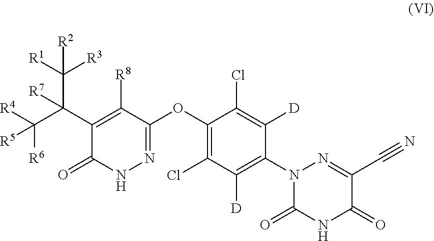
Wherein, R¹-R⁸ are each independently selected from H and D.

7. The compound according to claim 1 or an optical isomer, a pharmaceutically acceptable salt, a prodrug, a hydrate or a solvate thereof, characterized in that said compound is selected from any one of, but not limited to, the following compounds:

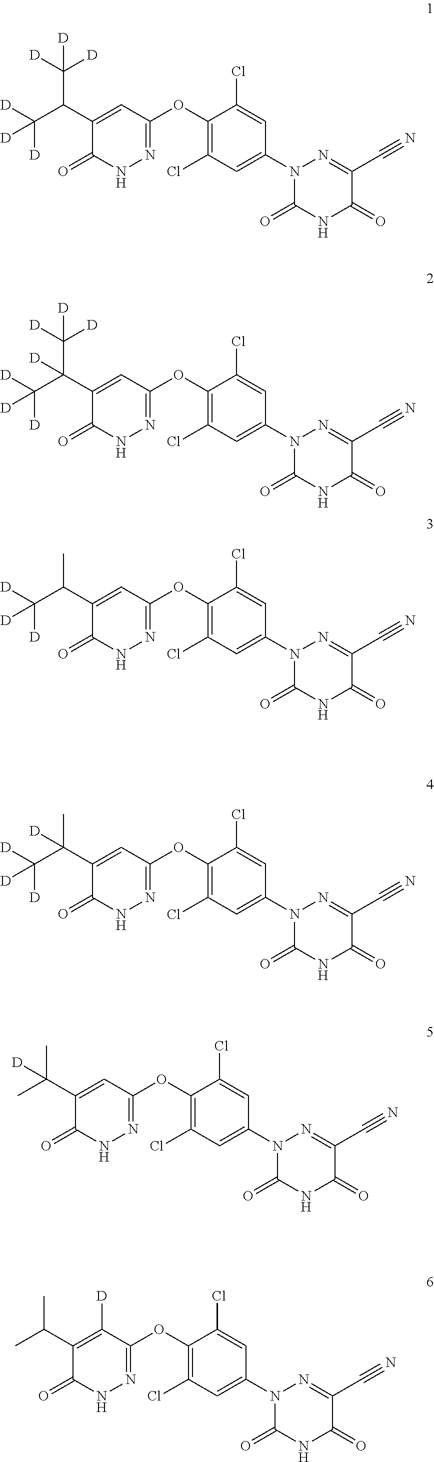 [SMILES: [2H]C([2H])([2H])C(c1cc(Oc2c(Cl)cc(-n3nc(C#N)c(=O)[nH]c3=O)cc2Cl)n[nH]c1=O)C([2H])([2H])[2H] ; [2H]C([2H])([2H])C([2H])(c1cc(Oc2c(Cl)cc(-n3nc(C#N)c(=O)[nH]c3=O)cc2Cl)n[nH]c1=O)C([2H])([2H])[2H] ; [2H]C([2H])([2H])C(C)c1cc(Oc2c(Cl)cc(-n3nc(C#N)c(=O)[nH]c3=O)cc2Cl)n[nH]c1=O ; [2H]C([2H])([2H])C([2H])(C)c1cc(Oc2c(Cl)cc(-n3nc(C#N)c(=O)[nH]c3=O)cc2Cl)n[nH]c1=O ; [2H]C(C)(C)c1cc(Oc2c(Cl)cc(-n3nc(C#N)c(=O)[nH]c3=O)cc2Cl)n[nH]c1=O ; [2H]c1c(Oc2c(Cl)cc(-n3nc(C#N)c(=O)[nH]c3=O)cc2Cl)n[nH]c(=O)c1C(C)C]

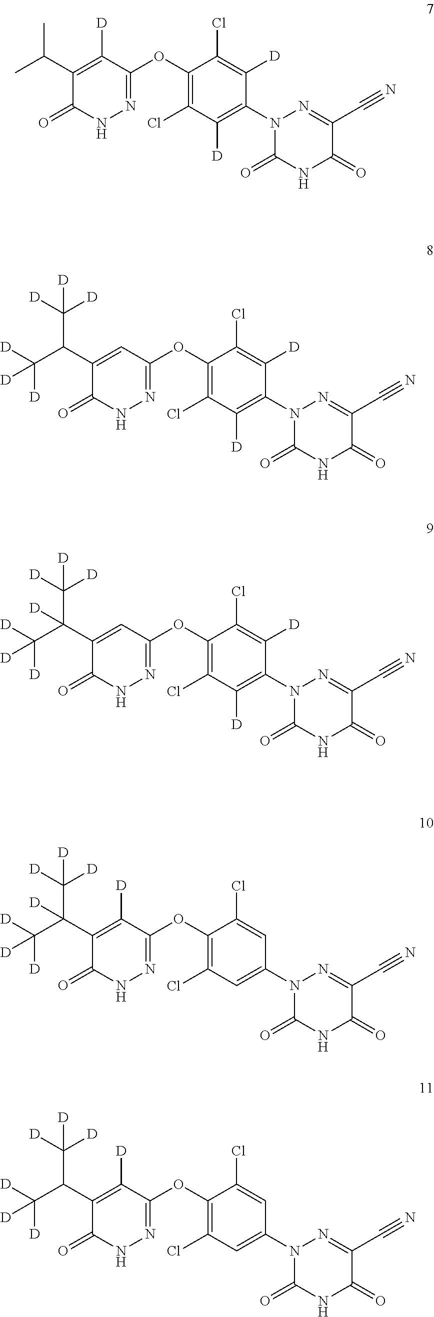

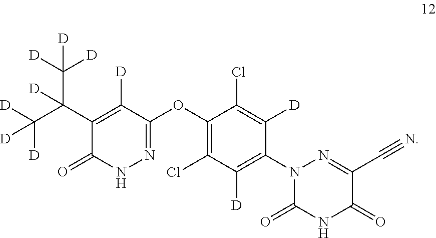 [SMILES: [2H]c1c(Cl)c(Oc2n[nH]c(=O)c(C([2H])(C([2H])([2H])[2H])C([2H])([2H])[2H])c2[2H])c(Cl)c([2H])c1-n1nc(C#N)c(=O)[nH]c1=O]

8. The use of the compound according to claim 1 or an optical isomer, a pharmaceutically acceptable salt, a prodrug, a hydrate or a solvate thereof in the preparation of the drugs for treating the indications suitable for THR-b agonists, reducing cholesterol, treating dyslipidemia, and treating non-alcoholic steatohepatitis and non-alcoholic fatty liver disease.

9. The use according to claim 8, characterized in that said drug is those for treatment of familial hypercholesterolemia, non-alcoholic steatohepatitis, and non-alcoholic fatty liver disease.

10. The use of the compound according to claim 1 or an optical isomer, a pharmaceutically acceptable salt, a prodrug, a hydrate or a solvate thereof in the preparation of THR-β agonist.

11. A drug for lowering cholesterol as well as treating dyslipidemia and non-alcoholic fatty liver, characterized in that it is a preparation obtained by using the compound according to claim 1 or an optical isomer, a pharmaceutically acceptable salt, a prodrug, a hydrate or a solvate thereof as active ingredients, with the addition of pharmaceutically acceptable excipients.
